(19) United States
(12) Patent Application Publication
(10) Pub. No.: US 2005/0222635 A1
(43) Pub. Date: Oct. 6, 2005

(54) Potency package two

(76) Inventor: Alexander A. Krakovsky, San Luis Obispo, CA (US)

Correspondence Address: John R. Ross, P.O. Box 2138, Del Mar, CA, 92014, US

(21) Appl. No.: 10/813,741

(22) Filed: Mar. 31, 2004

(51) Int. Cl. A61N 1/18

(52) U.S. Cl. 607/39

(57) ABSTRACT

A device and method for male impotence correction and female anorgasmy. An electronic stimulator with at least one pulse generator is implanted inside the body. At least one electrode is installed in the epidural space in the sacrum section of the spinal column and a conductor running under the user's skin electrically connects the electrode to the pulse generator. The stimulator is programmable and may be controlled from outside the body. Upon command initiated by the user or the user's lover the stimulator produces very short low-voltage electrical pulses in the sacrum section that are picked up by the nerves leading to the sex organs of the user, which stimulates arousal in the user's reproductive systems. The pulses are similar to the pulses generated by heart pacemakers. The present invention works on both males and females. In a preferred embodiment, the programmable electronic stimulator is implanted under the skin in the patient's back. Stimulation of the nerves coming out from the parasympathetic part of the spinal cord causes dilatation of the penile arteries in the male and in the clitoris arteries of the female, which results in an erection in the male and pre-orgasmic sensation in the female. In female, the stimulation of the sacral part of the spinal cord increases sexual desire and escalation to the level of orgasm. A preferred embodiment provides for emission stimulation. Emission is stimulated by electrical excitation of the sacral part of the spinal cord by increasing the voltage of the previous impulses. The device may be preprogrammed to set in motion the emission and ejaculation process at a predetermined time interval after the start of the erection process.

This invention relates to medical devices and methods and particularly to devices and methods for the correction of impotence in males and anorgasmy in females.

### BACKGROUND OF THE INVENTION

Sex Problems

Sexual performance in humans as well as in higher animals involves many functions. In males, there is erection, emission, ejaculation and orgasm. In females, there is initiation of sexual desire, escalation of the desire and orgasm. A wide variety of medical and psychological problems could interfere with one or more of these functions. The inability to achieve an erection is referred to as an erectile dysfunction or impotency. The inability to achieve an orgasm in females is referred to as anorgasmy. The principal methods presently used for male impotence correction and treatments include psychological and pharmacological treatments. Pharmacological treatments include noninvasive treatment (pills) and interventional treatment, which includes injection of vasoactive drugs into the penis. Surgical correction of impotence also exists. There is plastic surgery, prosthetic implantation and penile augmentation. There is no medical or surgical treatment that exists for anorgasmy in females at this time.

The Spinal Column and the Spinal Cord

The central nervous system in humans is comprised of the brain and the spinal cord. Nerve fibers running within the spinal cord provide communication between the brain and various parts of the body. Some actions (reflex actions) are mediated through nerve connections in the spinal cord without involving the brain. Nerves carrying signals to the central nervous system are called afferent neurons and nerves carrying signals away from the central nervous system are called efferent neurons. The spinal cord is contained within the spinal column (also called the vertebral column). The spinal column is comprised of 26 irregular bones connected into a flexible curved structure. These are grouped into five sections. From the top down these five sections are the cervical curvature with 7 vertebrae, the thoracic curvature with 12 vertebrae, the lumbar curvature with 5 vertebrae, the sacrum with 5 fused vertebrae and the coccyx with 4 fused vertebrae. C, T, L, and S numbers (i.e., C1through S5, numbered from the top of the cervical curvature to the bottom of the sacrum) identifies locations along the spinal column. See FIG. 4A. The spinal cord runs down from the brain through more than half of the spinal column. It terminates in or near the top of the lumbar curvature. Some nerves providing communication with the lower parts of the body continue on down through the spinal column. These nerves include the lumbar spinal nerves. The spinal cord and the lumbar spinal nerves are protected within the spinal column by a tough sheath called the spinal dural sheath. Just external to the spinal dura is a rather large epidural space filled with fat and a network of veins. The fat forms a protective padding around the spinal cord. See FIG. 4B. The dura and the epidural space extend well beyond the end of the spinal cord. Nerves branch out from the spinal column throughout its length to serve the various sections of the body. For the most part separate sets of nerves are provided for the left and right sides of the body.

How It Works

FIGS. 5 and 6 show reproductive features of a male human and a female human. Erection of the penis is generally a necessary prerequisite for penetration of the vagina. The stimuli for this reflex may involve: (1) the sacral segment of the spinal cord where the pudendal nerve is initiated, (2) peripheral pudendal nerve receptor stimulation (around the penis) or (3) mental stimulation. The stimuli exist in the forms of electrical signals. These signals are transmitted via nerves. The afferent signals are transmitted via the pelvic nerves (right or left side) to the sacral segment of the spinal cord. Efferent signals are transmitted via the pudendal nerves. The pudendal nerves provide electrical signals to the penis arteries and a very large number of small arteries inside the penis in corpora cavernosa and corpora spongiosa. These electrical signals result in dilatation of the arteries permitting an increase of blood flow into the penis, which has the effect of partially restricting the veins taking blood out of the penis. As a result, there is a rapid filling of the blood spaces in the corpora cavernosum and corpus spongiosum areas of the penis. The swelling erectile bodies within the penis press on blood vessels draining the penis slowing the drainage. This physiologic effect makes the penis rigid. Therefore, the net effect is erection. Emission is the movement of spermatozoa and secretions from the testes and other accessory glands into the urethra. This is entirely a reflex process not involving the brain. The afferent side of the reflex arc is initiated by touch receptors in the genital area such as a receptor in the gland penis. Electrical signals travels via the pelvic plexus, the sacral segment of the spinal cord and the pudendal nerve to stimulate sympathetic fibers, which stimulate the ductus deferens to slow pump sperm and seminal fluid into the urethra. Ejaculation is the propulsion of the semen out of the urethra. The same afferent paths are involved. Central connection are located in the sacral segment of the spinal cord. Afferent and efferent stimuli are conveyed by pars sympathetic fibers of the pelvic splenic nerves and the pudendal nerves. Ejaculation is caused by the rhythmic contraction of the bulbocavernosus muscle, while the internal vesicle sphincter closes, preventing retrograde ejaculation into the bladder.

Prior Art Patents

U.S. Pat. Nos. 5,246,015; 5,065,744 and 4,869,241 provide mechanical support for producing an erection. U.S. Pat. Nos. 5,236,904; 5,256,652 and 5,236,904 are the pharmaceutical type of impotence correction providing drugs administered to the penis. U.S. Pat. No. 5,454,840 issued to Applicant and one other describes a device and method for impotence correction. An electronic device is implanted inside the body. It is programmable and controllable from outside the body. The press of a button sends an electronic signal that initiates a process that simulates the body's natural reproductive processes. In a preferred embodiment, a programmable electronic device is implanted under abdominal muscle rectus. An electrical conductor is stitched to the surface of the pelvic splanchnic nerve. Stimulation of this nerve by a series of low voltage electrical pulses from the electronic device causes dilation of the penis arteries which results in an erection. The electronic device is controlled by a controller operated by the patient or his partner. Pat. No. '840 is incorporated herein by reference.

What is needed is an improved device and method for correcting dysfunctional impotence that simulates the natural processes of erection and ejaculation as closely as feasible.

### SUMMARY OF THE INVENTION

The present invention provides a device and method for male impotence correction and female anorgasmy. An electronic stimulator with at least one pulse generator is implanted inside the body. At least one electrode is installed in the epidural space in the sacrum section of the spinal column and a conductor running under the user's skin electrically connects the electrode to the pulse generator. The stimulator is programmable and may be controlled from outside the body. Upon command initiated by the user or the user's lover the stimulator produces very short low-voltage electrical pulses in the sacrum section that are picked up by the nerves leading to the sex organs of the user, which stimulates arousal in the user's reproductive systems. The pulses are similar to the pulses generated by heart pacemakers. The present invention works on both males and females. In a preferred embodiment, the programmable electronic stimulator is implanted under the skin in the patient's back. Stimulation of the nerves coming out from the parasympathetic part of the spinal cord causes dilatation of the penile arteries in the male and in the clitoris arteries of the female, which results in an erection in the male and pre-orgasmic sensation in the female. In female, the stimulation of the sacral part of the spinal cord increases sexual desire and escalation to the level of orgasm. A preferred embodiment provides for emission stimulation. Emission is stimulated by electrical excitation of the sacral part of the spinal cord by increasing the voltage of the previous impulses. The device may be preprogrammed to set in motion the emission and ejaculation process at a predetermined time interval after the start of the erection process. The controller also can be programmed to permit the patient or his/her partner to initiate the emission and ejaculation process. In the third preferred embodiment, a drug is administered from a stored chamber in the device. The drug is transmitted via a tube inside the spinal canal. As with the first two embodiments, control is in the hands of the user or the user's lover. Various combinations of the described above embodiments represent additional embodiments of the present invention.

### BRIEF DESCRIPTION OF DRAWINGS:

FIG. 1 shows a preferred embodiment installed in a user.

FIGS. 2A and 2B show the components of a preferred embodiment.

FIGS. 3A and 3B show preferred pulse shapes.

FIGS. 4A and 4B show features of a human spinal column and spinal cord.

FIGS. 5 and 5A and 6 show additional preferred embodiments of the present invention.

### DETAILED DESCRIPTION OF PREFERRED EMBODIMENTS

First Preferred Embodiment

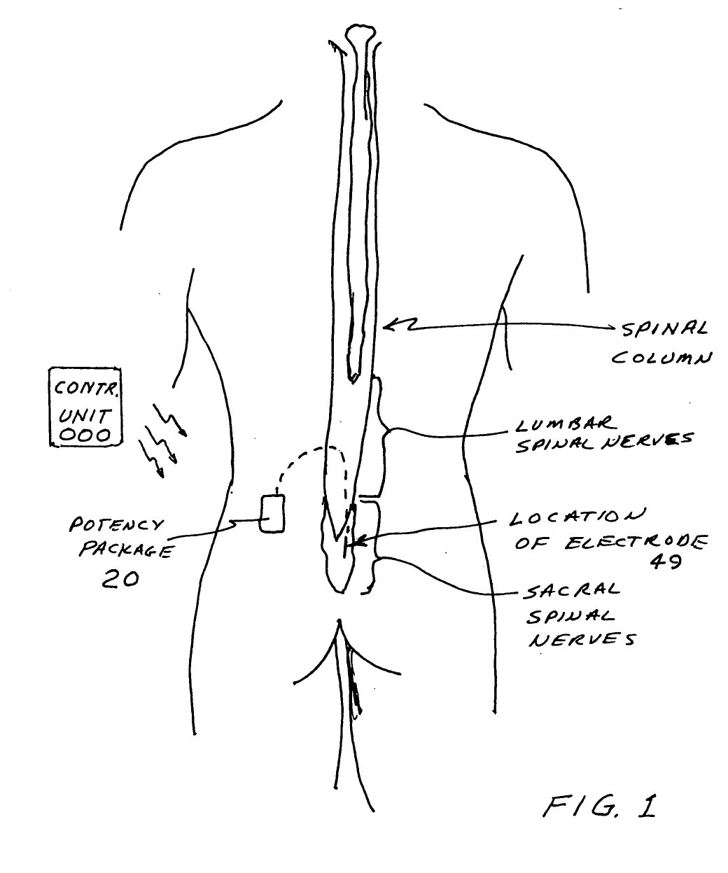

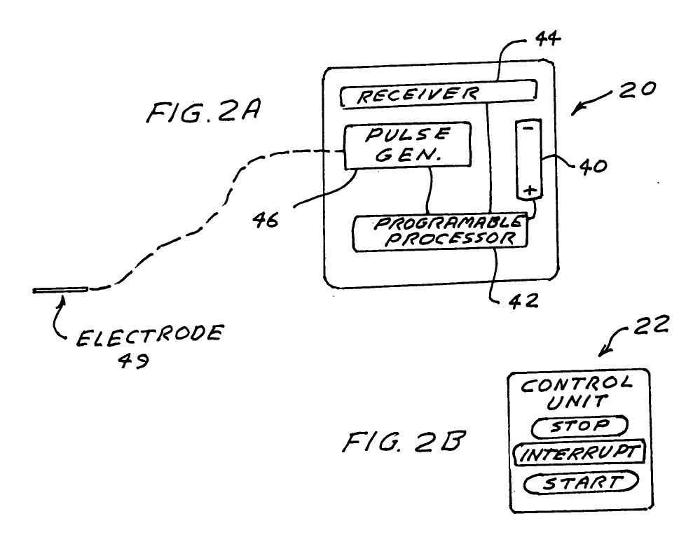

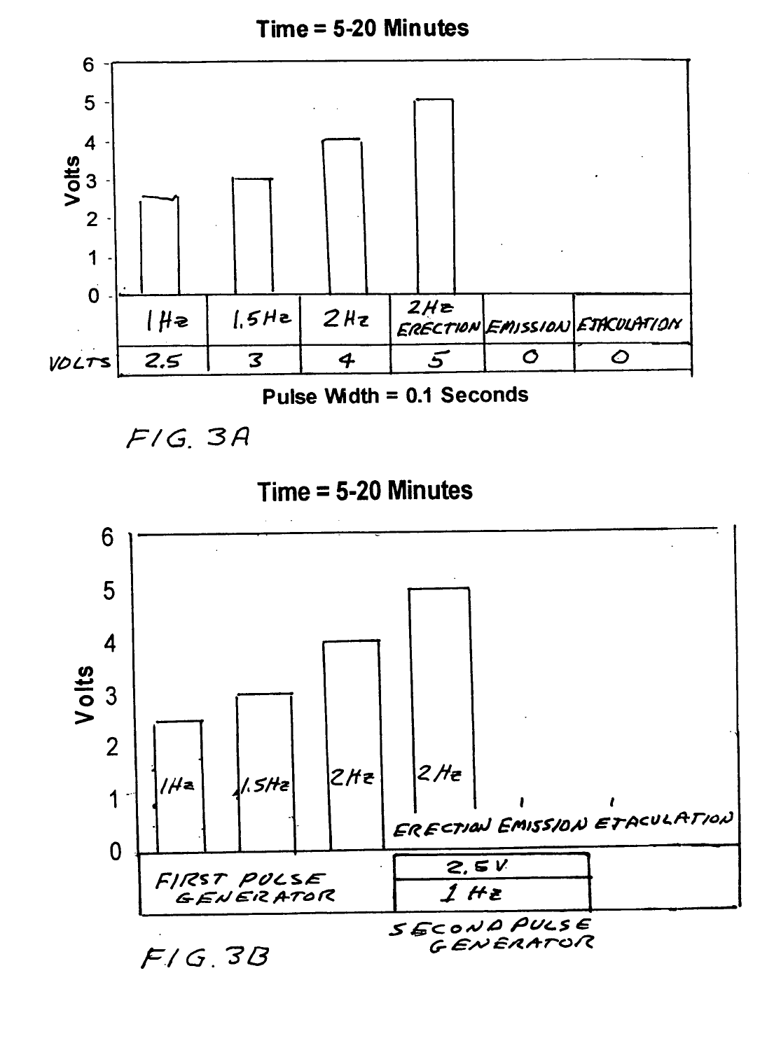

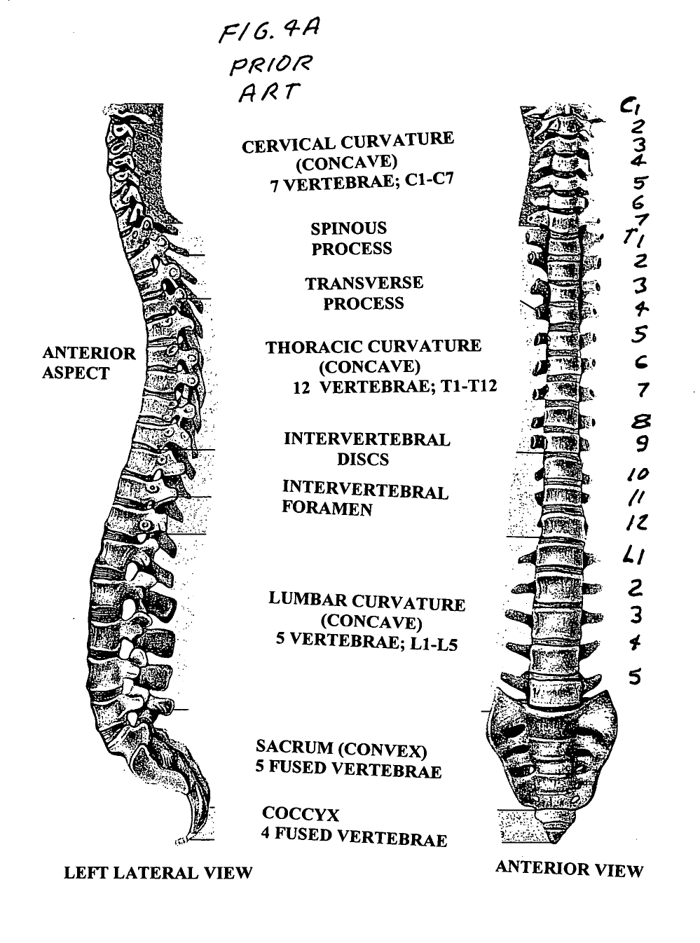

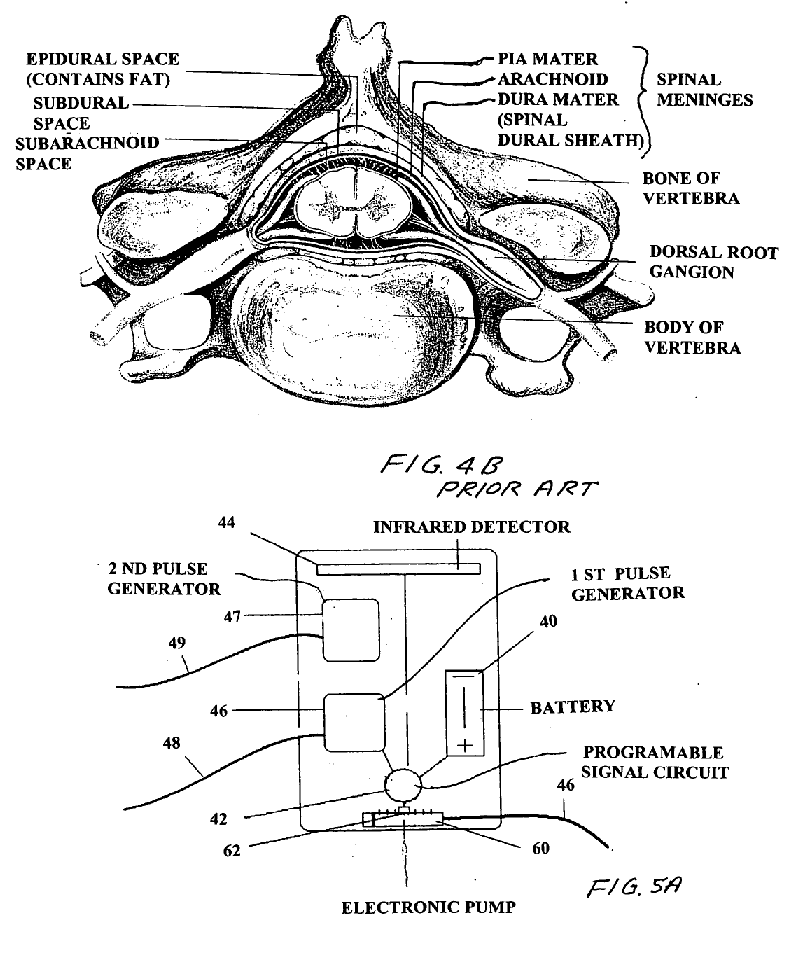

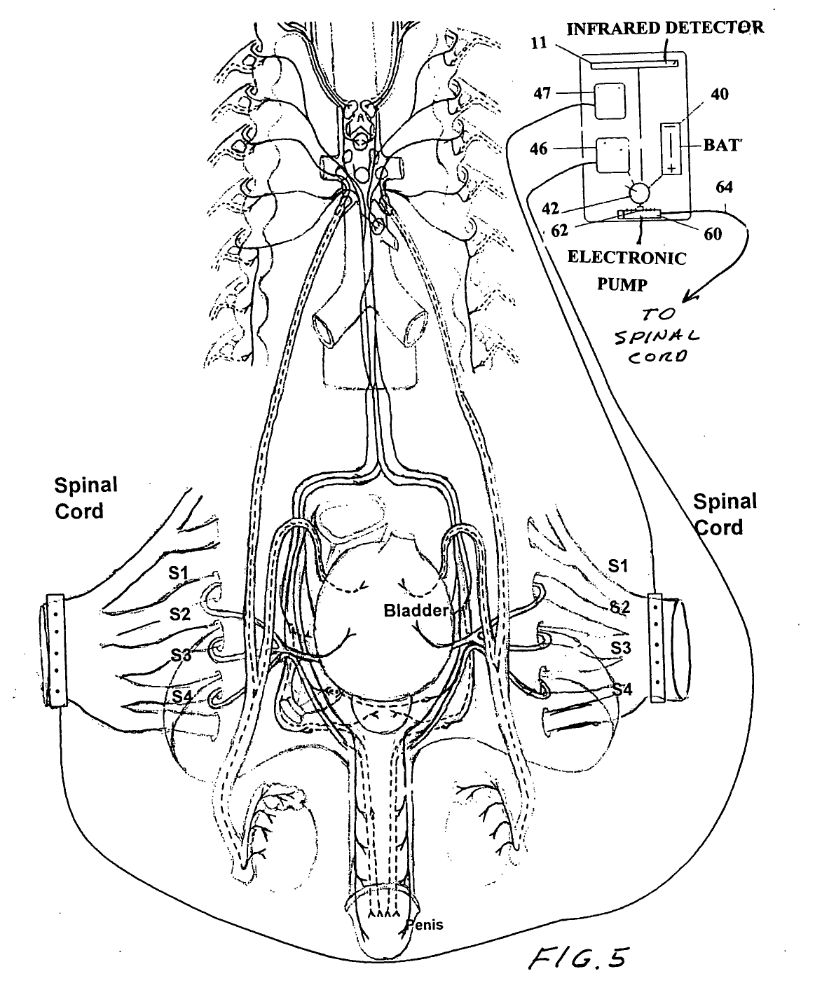

FIG. 1 shows a preferred embodiment of the present invention. An two-inch long thin electrode 49 located in the epidural space at the S2, S3 and S4 level of the sacrum on the right side of the sacral spinal nerves is connected by an electrical conductor running under the skin of the user to a stimulator device 20 implanted under the skin of the user in the top part of the user's buttox. FIG. 2A is a block diagram of the stimulator device, which the Applicant calls Potency Package Two. A preferred prototype embodiment comprises a modified commercial pacemaker Model 600AV manufactured by Semens and modified by the Applicant. The unit comprises a battery 40, a programmable signal circuit 42, a pulse generator 46 and a receiver antenna 44. The unit is controlled with an external control unit 22 shown at FIG. 2B. The unit comprises a start button, stop button and an interrupt button. The preferred sequence of pulses that should provide good results for many patients, is shown in FIGS. 3A and 3B. The package can be reprogrammed to change any of the parameters shown in FIGS. 3A and 3B, which are pulse height, pulse width, frequency, duration and sequence. The best program for each individual patient can be only determined by testing. These parameters such as number of pulses grouped, voltage, rates and pulse duration are well within the range available with the above potency package device. These parameters are among others that could be programmed, with the range of the device, using a commercially available pacemaker programmer such as Model #3CMHK850 supplied by MIFI and SMHK and also described in Patent '015 referred to in the Background Section. The programmer transmits programmed information via a pulse site and magnetic field generated in the programmer, to the electromagnetic detector in a programmable signal circuit of the implanted device. This device, shown in FIG. 2A, comprises only one electrode. Before permanent implantation of the device, every patient has to go through the testing trial. The trial designed to detect the ability of the stimulation to achieve an erection. The 2-inch long electrode runs from the epidural space at level S2, S3 and S4 of the sacral segment to under the patient's skin to connect the device. The procedure can be accomplished in any surgical center under monitored anesthesia care and local anesthesia. Personal surgical skill is required in order to install the device. The recommended electronic pulse series is shown in the FIG. 3A. If this series does not provide the desired effect, the doctor can vary the parameters. If an erection is produced by any of the tests, then the doctor continues the process and permanently installs the Potency Package Two as described above. If the doctor is unable to produce an erection in three days trial period, the doctor may choose not to proceed with the permanent implantation. In women patients, the clinical trial of the orgasm initiation stimulation contains the same approach in the same segment area in the sacral part of the spinal cord as in male patients. Instead of erection the doctor will be testing for pre-orgasm stimulation.

Surgery

The details of the surgery designed to provide the implantation of the electrode and device as described above is now described. A trained surgeon should surgically implant the Potency Package Two device and electrodes. The operation is very similar to the implantation of the heart pacemaker. The patient should be previously anesthetized, spontaneously breathing with the application of standard monitoring by American Society of Anesthesiology, which includes EKG, blood pressure, pulse monitor and oxygen by nasal cannula. The patient should be put in the prone position on the operating table and the site on the lumbar part of the patient's spine should be prepped and draped in sterile fashion. Local anesthesia should be applied to the lower lumbar area, and a 2-cm incision of the skin in the middle of the spine should be performed. The epidural space should be identified using loss of resistance technique with a Tuohy needle and fluoroscopic imaging. When the epidural space on the left side is identified, the electrode should be transmitted through the needle, and the needle should be withdrawn. The electrodes should enter to the epidural space at the level of lumbar part of the spinal cord and advance down to the sacral region. The proper position of the electrode should be verified under fluoroscopy. The electrode should be advanced to the sacral segment of the spinal cord at the level of S2, S3 and S4 on the left side. If an electrode is to be applied to the right side instead of or in addition to the left side the same loss or resistance technique and fluoroscopic imaging has to be applied on the right side of the spine to identify the epidural space on the right side. The right-side electrode should be positioning on the same level on the right side of the patient's sacral segment, S2, S3 and S4. The proper position of the second electrode should be confirmed by the fluoroscopy. After the proper positioning of the electrode (or electrodes) on the side or sides of the patient's spinal nerves in the epidural space at the level of the sacral segment of the spinal cord, a voltage signal (at a low range of available voltages) with the proper setting of the impulses should be applied through the electrode to the spinal cord. The patient should be asked what kind of sensation in his or her genitalia has been felt. If the male patient has established the initiation of the erection process, the electrodes should be affixed at those levels. If the initiation of the orgasm in the female has been identified by electrical stimulation, the electrodes also have to affix and sutured at the level, which was identified.

The stimulator should be installed in the buttock area just below the waist on the left or on the right side as the patient prefers, a 5-cm incision should be made after application of local anesthesia to this area. The pocket for the pacemaker should be made there. The tunnel from the initial part where the electrode has entered to the patient's body at the level of the lumbar part of the spinal cord should be transmitted to the pocket area. By using a screwdriver, the electrode is connected to the stimulator, and the skin over the incisions should be closed.

Second Preferred Embodiment

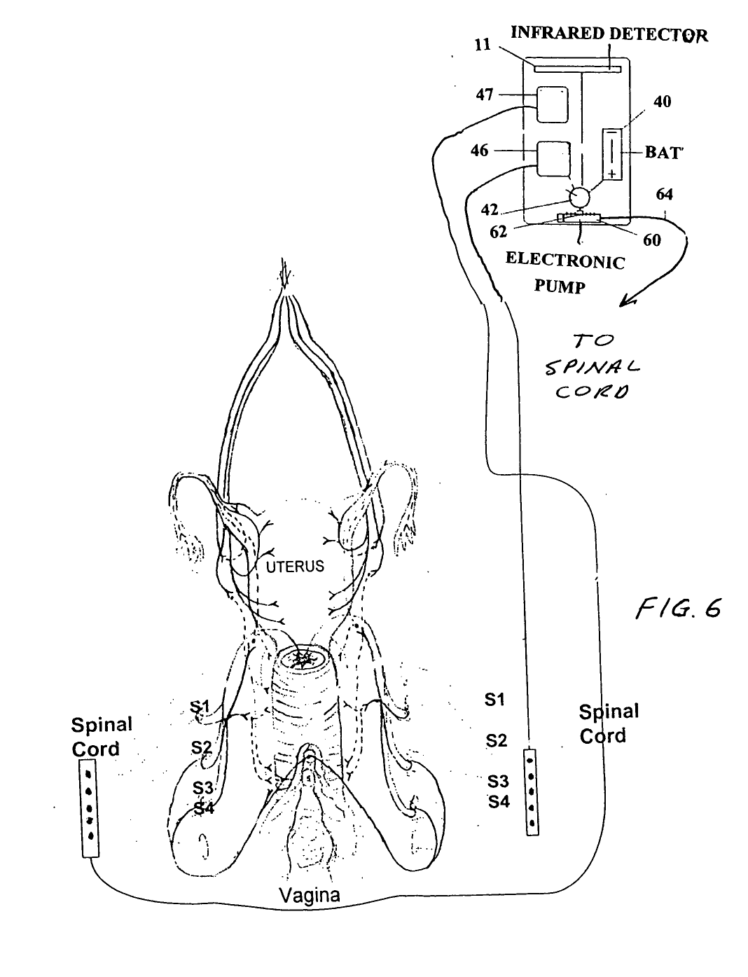

In a second preferred embodiment shown in FIG. 5, the stimulator contains a chamber 60 for storage within the body of a drug such papaverine and a small electronic pump 62 and a very thin tube for delivering of the drug to the spinal canal. The same result will be achieved with the delivering of the drug to the patient's body through the tube placed inside the spinal canal and deliver the drug in an on-and-off fashion to initiate the erection in the male. The delivery of the drug is initiated by an electronic signal transmitted by a hand-held transmitter controlled by the patient. For this alternative two electronic circuits are programmed as described above. The controller is programmed to deliver the drug at the time 0. A drug delivery chamber consists of plastic refillable containers, which is placed into hermetic chamber 62 as shown in FIG. 5A. The bottom of the chamber is a piston with a coil and electromagnetic step driver. The first step of the erection stimulation is a vasoactive drug (such as papaverine) delivered by sending an electrical potential to the driver. The driver pulls the coil into the electromagnet to apply a force squeezing the drug into the tube attached to the plastic container at one end and implanted into the spinal canal at the other end of the tube. Chamber 60 is refillable upon injection through the skin with a syringe injector. FIG. 6 shows this second embodiment as it is applied to a female.

Equipment

The potency package components can be standard off-the-shelf components. The components include: lithium battery, LBSAR5, made by SARATOF with a life-time of five to eight years, a pulse generator CLG445 made by MIFA, a receiver/transmitter MC145027 made by Motorola and IR remote control receiver 2338M made by AEG Corporation, and a fast IR Protodiode detector S113-11 made by Hamamatsu, IR remote control transmitter U327-M made by AEG Corporation, stepping motor 155ML microslide made Toshiba Corporation, Silicon tube catheter T5715 made by Dow Corning Silastic and elastimer Q7-4750 Silicon pack made by Dow Coming Silastic.

Other Embodiments

Various combinations of the above-described embodiments provide many other embodiments of the present invention. The fourth embodiment would provide for emission stimulation only. A fifth is drug only. The following table lists vasoactive drugs and recommended quantities.

| | | |
|---|---|---|
| Papaverine | 15 | mg |
| Fentolamine | 0.5 | mg |
| Prostaglandin E1 | 20 | mcg |
| Vasoactive intestinal polypeptide | 5 | mcg |

Providing more than one drug will design in additional embodiment. For example, a drug such as nitroglycerin releasing into the blood stream to providing protection for patients against heart attack during sex. The following table shows some drugs recommended for correction of the most common health conditions occurring during sexual intercourse.

| | |
|---|---|
| Arrhythmia | beta-blockers |
| Asthma | alpha-blockers |
| Angina | nitroglycerin |
| Hypertension | beta-blockers |

Using many different drugs that are known to induce erection provides additional embodiments. Also, there are many electronic pulse sequences, which would work well to produce erection, emission and ejaculation for many different patients in addition to the sequences described above. Skilled doctors will recognize that electrodes can be connected at different locations other than those described above. To correct the arterial circulatory problem of impotence, the arterial anastomosis should be performed at the same time with the Potency Pack Two implantation. Also the penis enlargement surgery and the penis lengthening surgery could be applied at the same time as the implementation of the electrodes for the correction of male impotence. The vascular problems related to peripheral vascular disease should preferably be treated with anastomosis between inferior hypogastric arteries and the central and dorsal artery and dorsal vein of the penis.

Diabetic Type Impotency Correction

To correct the diabetic circulatory problem of impotence, the arterial anastomosis should be performed to the penis at the same time with the Potency Package Two implantation. Anastomosis between the hypo-gastric artery and dorsal artery should be performed end-to-end or end-to-side. The penile vein of diabetic patients can usually provide blood flow and surgical correction is not required in most cases.

Male Enhancement Surgery

Potency Package Two could be implanted during the period of penis enlargement by Dermograph, or by Allograft. They are implanted inside the penis with the preservation of the corpora cavemosum and the corpus spongiosum. The dermal graft or allograft is implanted under the skin of the penis with fixation to the distal part of the glans penis and the proximal part of the shaft of the penis. The penile lengthening is done by dissection of the tendon, which is affixed to the base of the penis and the pubic bone. Increase in length of the penis usually is from 1½ inches to 3⅓ inches. Increase in girth of the penis depends upon the patient's preference. The average increase in penis size is 2½ inches to 4½ inches.

Treatment of Anorgasmia in Females

The surgical technique described above for males can be applied to treat anorgasmy in women. The stimulation of the sacral part of the spinal cord generates impulses through the pudendal nerve that supplies innervation to the inner and outer part of the vagina and the clitoris. This artificial stimulation leads to the generation of the impulses that provide the additional blood supply and lubrication to the vagina and increase of the arousal part of the sexual desire in women. The additional stimulation technique applied through the nervous system allows women to feel a higher level of sexual arousal, increase escalation of the orgasm and the actual achievement of the orgasm. The higher level of sexual arousal increases the ability to achieve orgasm in the majority of human subjects.

Animal Use

The present invention can be applied to many animals. It should be especially valuable for use with breeding animals such as prize bulls. It could also be used in a breeding program of captured members of endangered species in wild animals.

While the above description contains much specificity, the reader should not construe this as limitations of the scope of the invention, but merely as exemplification of preferred embodiments thereof. This skilled art will envision many other possible variations like another location for the stimulator, different types of electrodes and pacemakers, different voltages, amplitudes, pulse groups, repetition rate, pulse duration, remote control with more or less functions, fully automatic preprogrammed pacemaker without external controls, etc. Other elements in the woman's device might provide for an electrode for ureteral muscle contraction for urine incontinence correction during sexual intercourse. Additional electronic devices could be added to the potency package, such as a heart rate monitor a CO2 detector and a blood pressure detector. These devices could be designed to produce an alarm if the patient's data indicated that he or she is becoming too excited in the course of sex so that the patient an his partner can moderate their love making. Accordingly the reader is requested to determine the scope of the invention by the appended claims and their legal equivalence and not by the examples, which have been given.

1. A sex enhancement device comprising;
A) a simulator for providing stimulation to a sex organ of a patient, said stimulator comprising:
1) a stimulator package installed under skin of a said patient, said stimulator package comprising:
a) at least one pulse generator,
b) a receiver for receiving instructions from said patient or said patient's lover,
c) a programmable processor for controlling said at least one pulse generator to produce desired pulse shapes upon receipt of said instructions,
2) at least one electrode implanted in an epidural space in said patient's sacrum,
3) at least one electrical conductor located under said patient's skin and connecting said at least one electrode to said at least one pulse generator,
B) a controller for use by said patient or said patient's lover for transmitting instructions to said stimulator package.

2. The device as in claim 1 wherein said at least one electrode is implanted in as epidural space at levels S2, S3 and S4.

3. A device as in claim 1 wherein said at least one pulse generator is two pulse generators and said at least one electrode is two electrodes and said at least one electrical conductor is two electrical conductors.

4. The device as in claim 1 wherein said patient is a male human

5. The device as in claim 1 wherein said patient is a female human.

6. A device as in claim 1 wherein said stimulator package also comprises a storage vessel for the storage of vasoactive drug and said transmitting means for transmitting said vasoactive drug.

7. A device as in claim 1 wherein said stimulator package also comprises a storage vessel for the storage of vasoactive drug and said transmitting means for transmitting said vasoactive drug to said patient's sacrum.

8. A method of correcting impotency in a male animal comprising the steps of:
a. surgically implanting under the skin of said male animal a programmable electronic stimulator comprising an electrical pulse generator,
b. surgically implanting an electrode in a epidural region of said animal's sacrum,
c. providing an electrical connection between said pulse generator and said electrode,
d. providing an electronic control for controlling said stimulator for so as to produce erection in said male animal.

9. A method as in claim 8 wherein said male animal is a domesticated animal.

10. A method as in claim 8 wherein said male animal is a captured wild animal.

11. A method as in claim 8 wherein said male animal is a human.
